(19) United States
(12) Patent Application Publication
(10) Pub. No.: US 2022/0150087 A1
(43) Pub. Date: May 12, 2022

(54) SPEED TEST MEASUREMENT WITHOUT IMPACTING CUSTOMER SERVICES ON CABLE MODEM

(71) Applicant: ARRIS Enterprises LLC, Suwanee, GA (US)

(72) Inventors: Jagadeesh KARUTURI, Bangalore (IN); Harish SHANKARACHARI, Bangalore (IN); Harshita GANGWAR, Bangalore (IN); Kazuhiko TOMOMATSU, Naperville, IL (US); James William REYNOLDS, Brighton East (AU)

(21) Appl. No.: 17/474,353

(22) Filed: Sep. 14, 2021

Related U.S. Application Data
(60) Provisional application No. 63/110,725, filed on Nov. 6, 2020.

(51) Int. Cl. H04L 12/28

(52) U.S. Cl. CPC H04L 12/2801; H04L 12/2859; H04L 12/2892; H04L 12/2879

(57) ABSTRACT

A cable modem termination system (CMTS) for use with a modem test server, an external network, and a cable modem. The CMTS including a memory and a processor configured to execute instructions stored on memory to cause CMTS to: provide a downstream service flow to the cable modem; provide an upstream service flow to the cable modem; receive a speed test initiation request from the modem test server; provide a second downstream service flow to the cable modem, provide a second upstream service flow to the cable modem; receive a speed test query from the modem test server; transmit the speed test query to the cable modem via the second downstream service flow; receive a speed test response from the cable modem via the second upstream service flow; and transmit the speed test response to the modem test server.

### BACKGROUND

Embodiments of the present disclosure relate to speed tests on cable modems.

### SUMMARY

Aspects of the present disclosure are drawn to a cable modem termination system (CMTS) for use with a modem test server, an external network, and a cable modem, the modem test server being configured to transmit a speed test initiation signal and a speed test query, the cable modem being configured to transmit a speed test response, the CMTS including: a memory; and a processor configured to execute instructions stored on the memory to cause the CMTS to: provide a first downstream service flow to the cable modem, the first downstream service flow including first downstream data from the external network; provide a first upstream service flow from the cable modem, the first upstream service flow including first upstream data to be provided to the external network; receive the speed test initiation request from the modem test server; provide a second downstream service flow to the cable modem, the second downstream service flow being distinct from the first downstream service flow; provide a second upstream service flow to the cable modem, the second upstream service flow being distinct from the first upstream service flow; receive the speed test query from the modem test server; transmit the speed test query to the cable modem via the second downstream service flow; receive the speed test response from the cable modem via the second upstream service flow; and transmit the speed test response to the modem test server.

In some embodiments, the processor is configured to execute instructions stored on the memory to additionally cause the CMTS to stop providing the second downstream service flow and the second upstream service flow to the cable modem after the speed test response is transmitted to the modem test server.

In some further embodiments the processor is configured to execute instructions stored on the memory to additionally cause the CMTS to transmit a speed test result to the cable modem via the first downstream service flow.

In some embodiments, the processor is configured to execute instructions stored on the memory to additionally cause the CMTS to transmit a speed test result to the cable modem via the second downstream service flow.

Other aspects of the present disclosure are drawn to a method of using a cable modem termination system (CMTS) with a modem test server, an external network, and a cable modem, the modem test server being configured to transmit a speed test initiation signal and a speed test query, the cable modem being configured to transmit a speed test response, the method comprising: providing, via a processor configured to execute instructions stored on a memory, a first downstream service flow to the cable modem, the first downstream service flow including first downstream data from the external network; providing, via the processor, a first upstream service flow from the cable modem, the first upstream service flow including first upstream data to be provided to the external network; receiving, via the processor, the speed test initiation request from the modem test server; providing, via the processor, a second downstream service flow to the cable modem, the second downstream service flow being distinct from the first downstream service flow; providing, via the processor, a second upstream service flow to the cable modem, the second upstream service flow being distinct from the first upstream service flow; receiving, via the processor, the speed test query from the modem test server; transmitting, via the processor, the speed test query to the cable modem via the second downstream service flow; receiving, via the processor, the speed test response from the cable modem via the second upstream service flow; and transmitting, via the processor, the speed test response to the modem test server.

In some embodiments, the method includes stop providing, via the processor, the second downstream service flow and the second upstream service flow to the cable modem after the speed test response is transmitted to the modem test server.

In some further embodiments, the method further includes transmitting, via the processor, a speed test result to the cable modem via the first downstream service flow.

In some embodiments, the method includes transmitting, via the processor, a speed test result to the cable modem via the second downstream service flow.

Other aspects of the present disclosure are drawn to a non-transitory, computer-readable media having computer-readable instructions stored thereon, the computer-readable instructions being capable of being read by a CMTS for use with a modem test server, an external network, and a cable modem, the modem test server being configured to transmit a speed test initiation signal and a speed test query, the cable modem being configured to transmit a speed test response, wherein the computer-readable instructions are capable of instructing the CMTS to perform the method including: providing, via a processor configured to execute instructions stored on a memory, a first downstream service flow to the cable modem, the first downstream service flow including first downstream data from the external network; providing, via the processor, a first upstream service flow to the cable modem, the first upstream service flow including first upstream data to be provided to the external network; receiving, via the processor, the speed test initiation request from the modem test server; providing, via the processor, a second downstream service flow to the cable modem, the second downstream service flow being distinct from the first downstream service flow; providing, via the processor, a second upstream service flow to the cable modem, the second upstream service flow being distinct from the first upstream service flow; receiving, via the processor, the speed test query from the modem test server; transmitting, via the processor, the speed test query to the cable modem via the second downstream service flow; receiving, via the processor, the speed test response from the cable modem via the second upstream service flow; and transmitting, via the processor, the speed test response to the modem test server.

In some embodiments, the computer-readable instructions are capable of instructing the CMTS to perform the method further comprising stop providing, via the processor, the second downstream service flow and the second upstream service flow to the cable modem after the speed test response is transmitted to the modem test server.

In some further embodiments, the computer-readable instructions are also capable of instructing the CMTS to perform the method further including transmitting, via the processor, a speed test result to the cable modem via the first downstream service flow.

In some embodiments, the computer-readable instructions are capable of instructing the modem test server to perform the method further comprising transmitting, via the processor, a speed test result to the cable modem via the second downstream service flow.

Other aspects of the present disclosure are drawn to a cable modem for use with a CMTS, a modem test server, and an external network, the modem test server being configured to transmit a speed test initiation signal and a speed test query, the CMTS being configured to provide a first downstream service flow including first downstream data from the external network, a second downstream service flow including the speed test query, to provide a first upstream service flow including first upstream data to be provided to the external network, and to provide a second upstream service flow, the cable modem comprising: a memory; and a processor configured to execute instructions stored on the memory to cause the cable modem to: receive the first downstream service flow; transmit the first upstream data to the CMTS via the first upstream service flow to be provided to the external network; transmit a speed test initiation request to the CMTS via the first upstream service flow; receive the second downstream service flow from the CMTS; receive the speed test query from the CMTS via the second downstream service flow; and transmit a speed test response to the CMTS via the second upstream service flow.

Other aspects of the present disclosure are drawn to a method of using a cable modem with a CMTS, a modem test server, and an external network, the modem test server being configured to transmit a speed test initiation signal and a speed test query, the CMTS being configured to provide a first downstream service flow including first downstream data from the external network, a second downstream service flow including the speed test query, to provide a first upstream service flow including first upstream data to be provided to the external network, and to provide a second upstream service flow, the method including: receiving, via a processor configured to execute instructions stored on a memory, the first downstream service flow; transmitting, via the processor, the first upstream data to the CMTS via the first upstream service flow to be provided to the external network; transmitting, via the processor, a speed test initiation request to the CMTS via the first upstream service flow; receiving, via the processor, the second downstream service flow from the CMTS; receiving, via the processor, the speed test query from the CMTS via the second downstream service flow; and transmitting, via the processor a speed test response to the CMTS via the second upstream service flow.

Other aspects of the present disclosure are drawn to a non-transitory, computer-readable media having computer-readable instructions stored thereon, the computer-readable instructions being capable of being read by a cable modem for use with a CMTS, a modem test server, and an external network, the modem test server being configured to transmit a speed test initiation signal and a speed test query, the CMTS being configured to provide a first downstream service flow including first downstream data from the external network, to provide a second downstream service flow including the speed test query, to provide a first upstream service flow including first upstream data to be provided to the external network, and to provide a second upstream service flow, wherein the computer-readable instructions are capable of instructing the cable modem to perform the method including: receiving, via a processor configured to execute instructions stored on a memory, the first downstream service flow; transmitting, via the processor, the first upstream data to the CMTS via the first upstream service flow to be provided to the external network; transmitting, via the processor, a speed test initiation request to the CMTS via the first upstream service flow; receiving, via the processor, the second downstream service flow from the CMTS; receiving, via the processor, the speed test query from the CMTS via the second downstream service flow; and transmitting, via the processor a speed test response to the CMTS via the second upstream service flow.

Other aspects of the present disclosure are drawn to a cable modem for use with a cable modem termination system (CMTS), a modem test server, and an external network. The CMTS is configured to provide a first downstream service flow including first downstream data from the external network, to provide a second downstream service flow, to provide a first upstream service flow including first upstream data to be provided to the external network, and to provide a second upstream service flow. The cable modem includes: a memory; and a processor configured to execute instructions stored on the memory to cause the cable modem to: receive the first downstream service flow; transmit the first upstream data to the CMTS via the first upstream service flow to be provided to the external network; receive the second downstream service flow from the CMTS; transmit a speed test query to the modem test server via the CMTS and via the second upstream service flow; and receive a speed test response from the modem test server via the CMTS and via the second downstream service flow.

In some embodiments, the processor is configured to execute instructions stored on the memory to additionally cause the cable modem to transmit a speed test response to the CMTS via the second upstream service flow.

In some embodiments, the processor is configured to execute instructions stored on the memory to additionally cause the cable modem to transmit a speed test initiation request to the CMTS via the first upstream service flow.

Other aspects of the present disclosure are drawn to a method of using a cable modem with a cable modem termination system (CMTS), a modem test server, and an external network. The CMTS is configured to provide a first downstream service flow including first downstream data from the external network, to provide a second downstream service flow, to provide a first upstream service flow including first upstream data to be provided to the external network, and to provide a second upstream service flow. The method includes: receiving, via a processor configured to execute instructions stored on a memory, the first downstream service flow; transmitting, via the processor, the first upstream data to the CMTS via the first upstream service flow to be provided to the external network; receiving, via the processor, the second downstream service flow from the CMTS; transmitting, via the processor a speed test query to the modem test server via the CMTS and via the second upstream service flow; and receiving, via the processor, a speed test response from the modem test server via the CMTS and via the second downstream service flow.

In some embodiments, the method further includes transmitting, via the processor, a speed test response to the CMTS via the second upstream service flow.

In some embodiments, the method further includes transmitting, via the processor, a speed test initiation request to the CMTS via the first upstream service flow.

Other aspects of the present disclosure are drawn to a non-transitory, computer-readable media having computer-readable instructions stored thereon, the computer-readable instructions being capable of being read by a cable modem for use with a cable modem termination system (CMTS), a modem test server, and an external network. The CMTS is configured to provide a first downstream service flow including first downstream data from the external network, to provide a second downstream service flow, to provide a first upstream service flow including first upstream data to be provided to the external network, and to provide a second upstream service flow. The computer-readable instructions are capable of instructing the cable modem to perform the method including: receiving, via a processor configured to execute instructions stored on a memory, the first downstream service flow; transmitting, via the processor, the first upstream data to the CMTS via the first upstream service flow to be provided to the external network; receiving, via the processor, the second downstream service flow from the CMTS; transmitting, via the processor a speed test query to the modem test server via the CMTS and via the second upstream service flow; and receiving, via the processor, a speed test response from the modem test server via the CMTS and via the second downstream service flow.

In some embodiments, the computer-readable instructions are capable of instructing the cable modem to perform the method further including transmitting, via the processor, a speed test response to the CMTS via the second upstream service flow.

In some embodiments, the computer-readable instructions are capable of instructing the cable modem to perform the method further including transmitting, via the processor, a speed test initiation request to the CMTS via the first upstream service flow.

### BRIEF SUMMARY OF THE DRAWINGS

The accompanying drawings, which are incorporated in and form a part of the specification, illustrate example embodiments and, together with the description, serve to explain the principles of the present disclosure. In the drawings:

FIG. 1A illustrates structural components implementing a communication infrastructure at a time t₀;

FIG. 1B illustrates structural components implementing a communication infrastructure at a time t₁;

FIG. 2A illustrates an exploded view of a network node at time t₀;

FIG. 2B illustrates an exploded view of a network node at time t₁;

FIG. 3 illustrates a method of optimizing an internet speed test by creating separate upstream and downstream service flows to and from a modem test server in accordance with aspects of the present disclosure;

FIG. 4A illustrates structural components implementing a communication infrastructure at a time t₂;

FIG. 4B illustrates structural components implementing a communication infrastructure at a time t₃;

FIG. 4C illustrates structural components implementing a communication infrastructure at a time t₄;

FIG. 5 illustrates an exploded view of a modem test server, a CMTS, a network node, and a cable modem;

FIG. 6 illustrates an exploded view of a network node at a time t₃; and

FIG. 7 illustrates structural components implementing a communication infrastructure at a time t₅.

### DETAILED DESCRIPTION

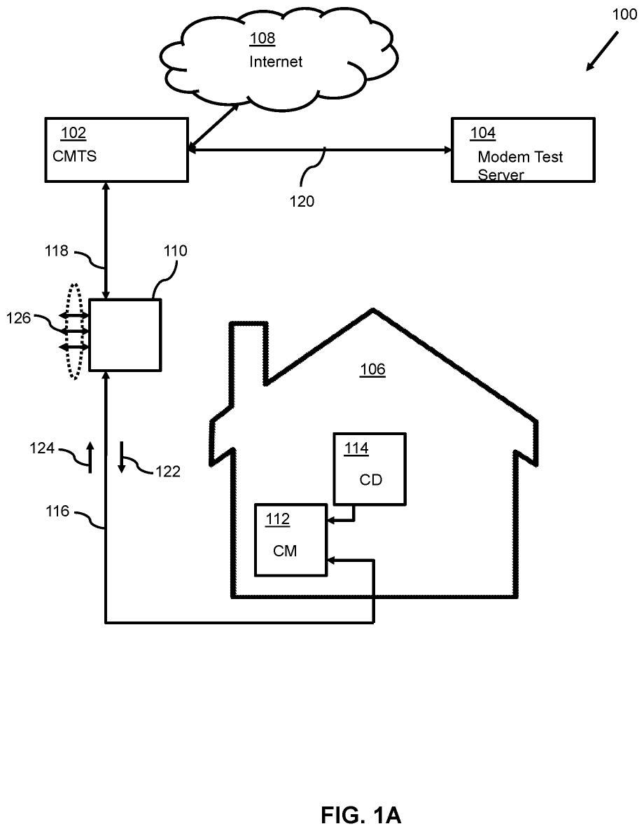

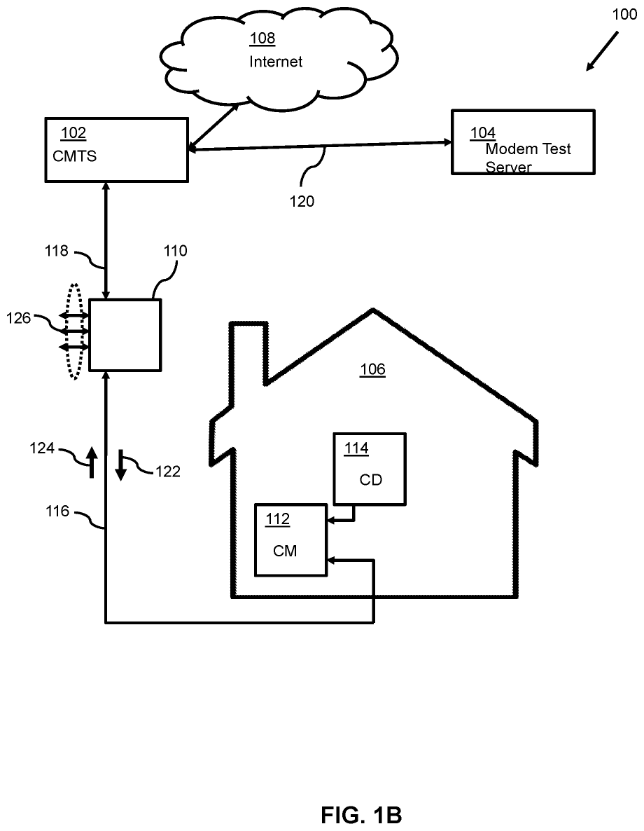

FIG. 1A illustrates structural components implementing a communication infrastructure 100 at a time to.

As shown in the figure, communication infrastructure 100 includes: a CMTS 102, a modem test server 104; a residence 106 which includes one cable modem 112 and a client device 114; internet 108; a network node 110; communication channels 116, 118, and 120; a downstream service flow 122, an upstream service flow 124, and a plurality of service flows 126. Cable modem 112 can communicate with modem test server 104 by way of network node 110 and CMTS 102. Network node 110 provides a plurality of service flows 126 to multiple home networks.

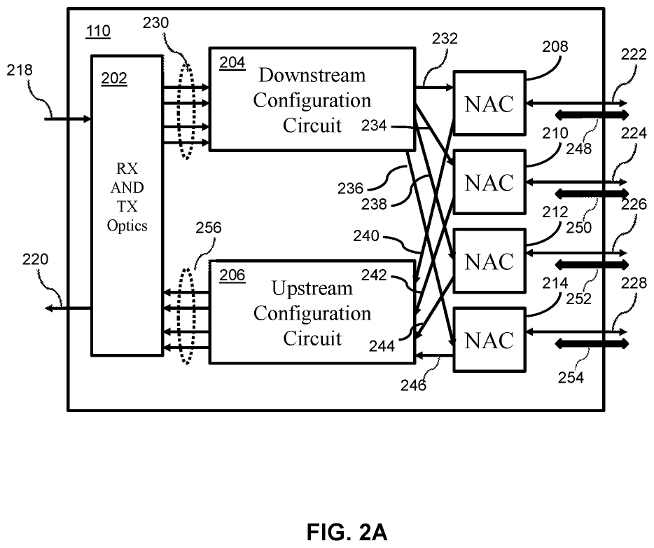

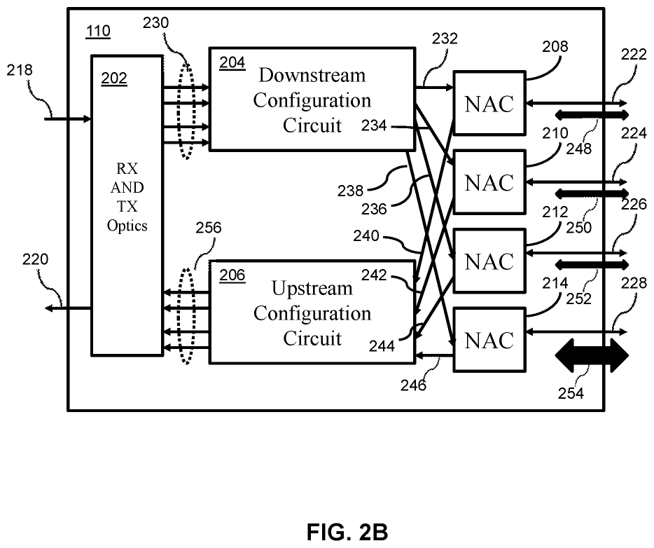

FIG. 2A illustrates an exploded view of network node 110 as shown in FIGS. 1A at time to.

As shown in FIG. 2A, network node 110 includes an optical communication component 202, a downstream configuration circuit 204, an upstream configuration circuit 206, a network access component (NAC) 208, a NAC 210, a NAC 212, and a NAC 214. Network node 110 communicates with CMTS 102 via downstream service flow 218 and upstream service flow 220. For simplicity, downstream service flow 218 and upstream service flow 220 are shown as a communication line 118 in FIG. 1A.

FIG. 1B illustrates structural components implementing a communication infrastructure 100 at a time

As shown in the figure, communication infrastructure 100 includes a CMTS 102, a modem test server 104, a residence 106 which includes one cable modem 112 and a client device 114, internet 108, a network node 110, communication channels 116, 118, and 120, a downstream service flow 122, an upstream service flow 124, and a plurality of service flows 126. Cable modem 112 can communicate with modem test server 104 by way of network node 110 and CMTS 102. Network node 110 provides a plurality of service flows 126 to multiple home networks.

A speed test checks the upload speed, which is the speed at which data is uploaded from cable modem 112 to Internet 108 and the download speed, which is the speed at which data is downloaded from Internet 108 to cable modem 112. To have a common measuring distance, as Internet 108 is worldwide ranging, the upload speed is actually measured by determining the time for which speed data packets from cable modem 112 pass through CMTS 102 so as to have access to Internet 108. This is performed by having modem test server 104 disposed at the output of CMTS 102. Similarly, the download speed is actually measured by determining the time for which speed data packets from outside of CMTS 102 pass through CMTS 102 and reach cable modem 112. Again, this is accomplished by having modem test server 104 disposed at the output of CMTS 102. Accordingly, upload speed is measured by measuring speed data packets from cable modem 112, through CMTS 102, and to modem test server 104, whereas the download speed is measured by measuring speed data packets from modem test server 104, through CMTS 102 and to cable modem 112.

In some cases, a homeowner might want to run a speed test. In this case, the speed test is initiated by cable modem 112. In other cases, a service provider may be required to provide speed tests to assure an advertised level of service. In these cases, CMTS 102 may initiate a speed test. In any event, for purposes of discussion presume that a speed test is being conducted on cable modem 112. At time t₁, additional bandwidth is needed to conduct an internet speed test.

This will be described with greater reference to FIG. 2B.

FIG. 2B illustrates an exploded view of network node 110 as shown in FIG. 1B at time t₁.

At time t₁, additional bandwidth is needed to conduct an internet speed test for residence 106. As shown in the figure, service flow 254 is larger than service flows 248, 250, and 252. CMTS 102 will instruct network node 110 to increase the bandwidth of service flow 254 for communication line 116. This causes a major issue at the home network level since increasing the service flow on communication line 116 to residence 106 only addresses the bandwidth increase request in general and does not address the request which is specifically for a traffic bandwidth increase. At the home network level, traffic flow now has to compete with residential traffic for more bandwidth so its quality of service is not always the same.

What is needed is a system and method for bypassing home traffic flow when conducting a speed test.

A system and method in accordance with the present disclosure bypasses home traffic flow when conducting a speed test.

In accordance with the present disclosure, the present disclosure allows a modem test server to create a second data flow, parallel to the existing home network data flow. Speed in this instance is a measure of how long it takes for a certain number of bytes to be uploaded or downloaded in a specific time frame. In some embodiments, the modem test server is within the CMTS. The second data flow is only used to conduct internet speed tests. The modem test server will send a speed test query through the CMTS to the cable modem to find the download speed, and the speed test response is sent back through the CMTS to the modem test server to find the upload speed. Once the upload and download speed is found, the second data flow is terminated.

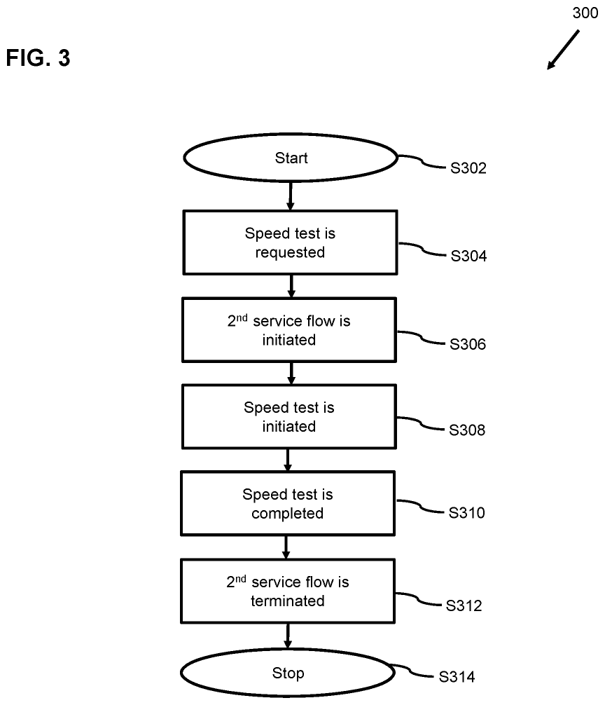

An example system and method for bypassing home traffic flow when conducting an internet speed test in accordance with aspects of the present disclosure will now be described in greater detail with reference to FIGS. 3-7.

FIG. 3 illustrates a method 300 of optimizing an internet speed test by creating separate upstream and downstream service flows to and from a modem test server in accordance with aspects of the present disclosure.

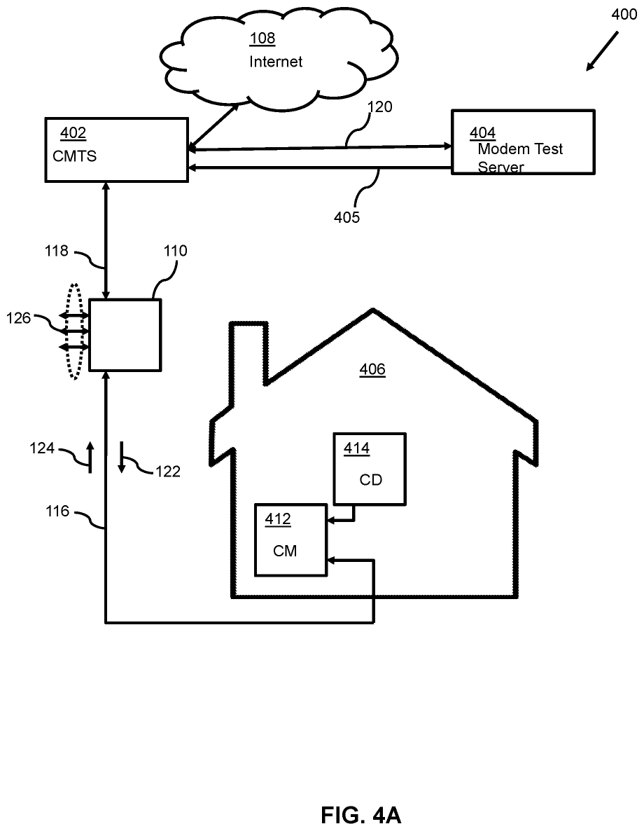

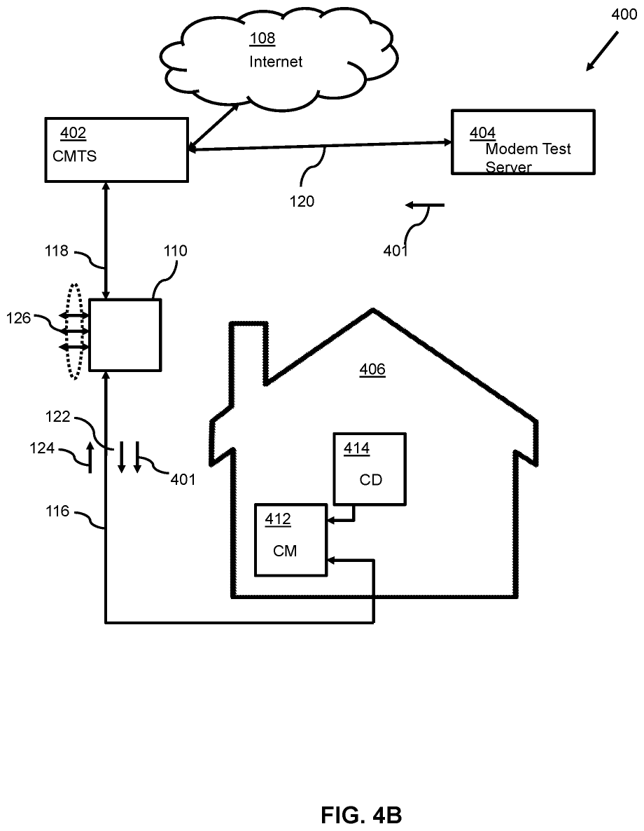

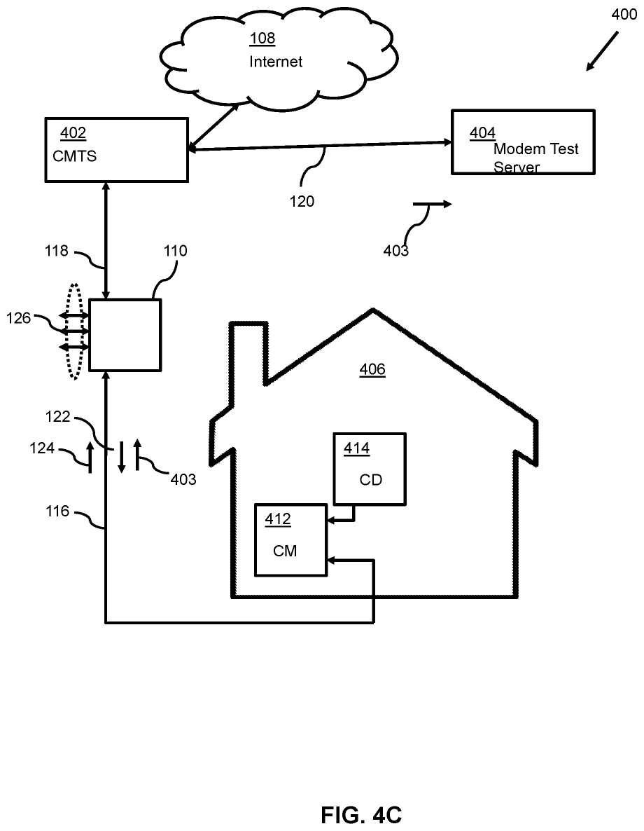

As shown in the figure, method 300 starts (S302), and a speed test is requested (S304). This will now be discussed in greater detail with reference to FIG. 4A.

FIG. 4A illustrates structural components implementing a communication infrastructure 400 at a time t₂ .

As shown in the figure, communication infrastructure 400 includes a CMTS 402, a modem test server 404, a speed test request 405, a residence 406 which includes one cable modem 412 and a client device 414, internet 108, a network node 110, communication channels 116, 118, and 120, a downstream service flow 122, an upstream service flow 124, and a plurality of service flows 126. Cable modem 412 can communicate with modem test server 404 by way of network node 110 and CMTS 402. Network node 110 provides a plurality of service flows 126 to multiple home networks.

As illustrated in FIG. 4A, modem test server 404 decides to conduct a test on residence 406. To do so, modem test server 404 will begin by sending a speed test request 405 to CMTS 402.

Returning to FIG. 3, after a speed test is requested (S304), a second service flow is initiated (S306). For example, returning to FIG. 4A, assume modem test server 404 is conducting an interne speed test on residence 406. This will be described in greater detail with reference to FIGS. 4B-6

FIG. 4B illustrates structural components implementing a communication infrastructure 400 at a time t3.

In addition to the components of FIG. 4A, FIG. 4B also includes a downstream service flow 401, which is distinct from downstream service flow 122.

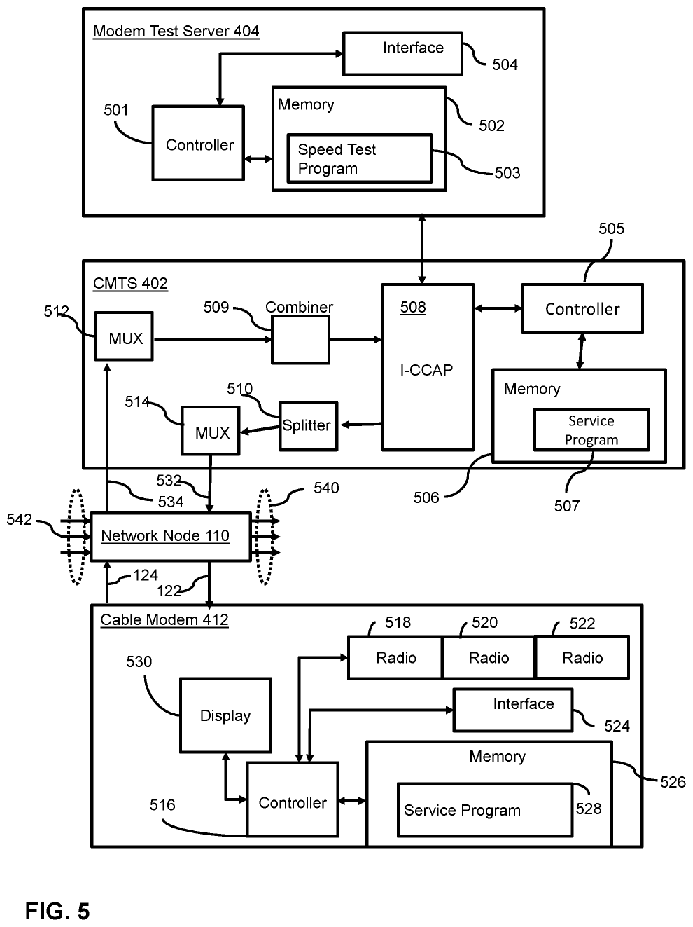

FIG. 5 illustrates an exploded view of modem test server 404, CMTS 402, network Node 410, and cable modem 412.

As shown in FIG. 5, modem test server 404 includes: a controller 501, a memory 502, which has stored therein a speed test program 503, and an interface circuit 504.

In this example, controller 501, memory 502, and interface circuit 504 are illustrated as individual devices. However, in some embodiments, they may be combined as a unitary device. Whether as individual devices or as combined devices, controller 501, memory 502, and interface circuit 504 may be implemented as any combination of an apparatus, a system and an integrated circuit. Further, in some embodiments, at least one of controller 501, memory 502, and interface circuit 504 may be implemented as a computer having non-transitory computer-readable media for carrying or having computer-executable instructions or data structures stored thereon. Such non-transitory computer-readable recording medium refers to any computer program product, apparatus or device, such as a magnetic disk, optical disk, solid-state storage device, memory, programmable logic devices (PLDs), DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, includes compact disc (CD), laser disc, optical disc, digital versatile disc (DVD), floppy disk and Blu-ray disc. Combinations of the above are also included within the scope of computer-readable media. For information transferred or provided over a network or another communications connection (either hardwired, wireless, or a combination of hardwired or wireless) to a computer, the computer may properly view the connection as a computer-readable medium. Thus, any such connection may be properly termed a computer-readable medium. Combinations of the above should also be included within the scope of computer-readable media.

Controller 501 can include a dedicated control circuit, CPU, microprocessor, etc. Controller 501 controls the circuits of modem test server 404.

Memory 502 can store various programming, user content, and data such as speed test program 503.

As will be described in greater detail below, in some embodiments, controller 501 is configured to execute instructions stored in speed test program 503 to enable modem test server 404 to transmit a speed test initiation signal and a speed test query.

As will be described in greater detail below, in some embodiments, controller 501 is configured to execute instructions stored in speed test program 503 to enable modem test server 404 to transmit a speed test query and receive a speed test response.

As will be described in greater detail below, in some embodiments, controller 501 is configured to execute instructions stored in speed test program 503 to enable modem test server 404 to determine a speed test result based on a speed test query and a speed test response.

As will be described in greater detail below, in some embodiments, controller 501 is configured to execute instructions stored in speed test program 503 to enable modem test server 404 to transmit a speed test result to CMTS 402.

As will be described in greater detail below, in some embodiments, controller 501 is configured to execute instructions stored in speed test program 503 to enable modem test server 404 to transmit a speed test result to cable modem 412 by way of CMTS 402.

CMTS 402 includes: a controller 505, a memory 506, which has stored therein a service program 507, an I-CCAP 508, a combiner 509, a splitter 510, an upstream MUX 512, and a downstream MUX 514.

In this example, controller 505, memory 506, I-CCAP 508, combiner 509, splitter 510, upstream MUX 512, and downstream MUX 514 are illustrated as individual devices. However, in some embodiments, they may be combined as a unitary device. Further, in some embodiments, controller 505 and memory 506 may be implemented as a computer having tangible computer-readable media for carrying or having computer-executable instructions or data structures stored thereon.

Controller 505 may be implemented as a hardware processor such as a microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for controlling the operation and functions of CMTS 402 in accordance with the embodiments described in the present disclosure.

As will be described in greater detail below, in some embodiments, controller 505 is configured to execute instructions stored in service program 507 to enable CMTS 402 to: provide a first downstream service flow to the cable modem, the first downstream service flow including first downstream data from the external network; provide a first upstream service flow to the cable modem, the first upstream service flow including first upstream data to be provided to the external network; receive the speed test initiation request from the modem test server; provide a second downstream service flow to the cable modem, the second downstream service flow being distinct from the first downstream service flow; provide a second upstream service flow to the cable modem, the second upstream service flow being distinct from the first upstream service flow; receive the speed test query from the modem test server; transmit the speed test query to the cable modem via the second downstream service flow; receive the speed test response from the cable modem via the second upstream service flow; and transmit the speed test response to the modem test server.

As will be described in greater detail below, in some embodiments, controller 505 is configured to execute instructions stored in service program 507 to additionally cause CMTS 402 to stop providing the second downstream service flow and the second upstream service flow to cable modem 412 after the speed test response is transmitted to modem test server 404.

As will be described in greater detail below, in some embodiments, controller 505 is configured to execute instructions stored in service program 507 to additionally cause CMTS 402 to transmit a speed test result to the cable modem via the first downstream service flow.

As will be described in greater detail below, in some embodiments, controller 505 is configured to execute instructions stored in service program 507 to additionally cause CMTS 402 to transmit a speed test result to the cable modem via the second downstream service flow.

Memory 506 can store various programming, user content, and data as service program data 507.

I-CCAP 508 is a platform which integrates multiple functions including a Data Over Cable Service Interface Specification (DOCSIS) cable modem termination system (CMTS), broadcast video Quadrature Amplitude Modulation (QAM) standard, Video On-Demand (VOD) Edge QAMs (EQAMS), and Set-top Box (STB) Out-Of-Band control. I-CCAP 508 provides broadband data for each cable modem, for example, cable modem 412, as an RF signal with a spectrum of frequencies.

Combiner 509 may be implemented as a hardware processor such as a microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for receiving RF signals from upstream MUX 512, combining and splitting RF signals as necessary, and transmitting them as a single RF signal to I-CCAP 508.

Splitter 510 may be implemented as a hardware processor such as a microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for receiving multiple RF signals from I-CCAP 508 plus other RF signals from other sources in the broadband data provider facility; and then splitting and combining the RF signals to effectively route them to downstream MUX 514.

Upstream MUX 512 may be implemented as a hardware processor such as a microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for receiving an optical signal from network node 110 and then de-multiplexing and receiving (RX) the optical signal into separate RF signals for transmission to combiner 509.

Downstream MUX 514 may be implemented as a hardware processor such as a microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for receiving the RF signals from splitter 510 and then optically transmitting (TX) and multiplexing the RF signals together on multiple wavelengths for transmission as a single optical signal to network node 110. Therefore, returning to FIGS. 4A-C, the signal from downstream MUX 514 on communication line 118 is a collection of one or more service flow data signals.

Returning to FIG. 5 network node 110 provides a plurality of service flows to multiple subscribers (or home networks) respectively. The plurality of service flows from network node 110 includes: downstream service flow group 540 and upstream service flow group 542. For example, network node 110 connects to CMTS 402 via communication line 118 (as illustrated in FIGS. 4A-C), where it provides a group of service flows indicated as upstream service flow group 534 and downstream service flow group 532.

Downstream service flow group 532 includes a plurality of distinct service flows that are split into individual service flows to be provided to different residences, which in this example includes a plurality of downstream service flows 540 to be provided to a plurality of respective cable modems at other residences (not shown) and a downstream service flow 122 to be provided to cable modem 412 via communication line 116 (as illustrated in FIGS. 4A-C).

Upstream service flow group 534 includes a plurality of distinct service flows that are multiplexed from individual service flows that are provided from different residences, which in this example includes a plurality of upstream service flows 542 to be provided from a plurality of respective cable modems at other residences (not shown) and an upstream service flow 124 to be provided from cable modem 412 via communication line 116 (as illustrated in FIGS. 4A-C).

Cable modem 412 includes a controller 516, a memory 526, which has stored therein a service program 528, a radio 518, 520, and 522, an interface 524, and a display 530.

In this example, controller 516, memory 526, radios 518, 520, and 522, interface 524, and display 530 are illustrated as individual devices. However, in some embodiments, they may be combined as a unitary device. Further, in some embodiments, controller 516 and memory 526 may be implemented as a computer having tangible computer-readable media for carrying or having computer-executable instructions or data structures stored thereon.

Controller 516 may be implemented as hardware circuitry such as a dedicated control circuit, CPU, a hardware processor such as a microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for controlling the operation and functions of the Wi-Fi extender 112 in accordance with the embodiments described in the present disclosure. Controller 516 controls the circuits of cable modem 412.

Memory 526 can store various programming, user content, and data, such as service program 528.

As will be discussed in more detail below, in some embodiments, service program 528 includes instructions that may be used by controller 516 to cause cable modem 412 to receive the first downstream service flow, transmit the first upstream data to the CMTS via the first upstream service flow to be provided to the external network, transmit a speed test initiation request to the CMTS via the first upstream service flow, receive a second downstream service flow from the CMTS, receive the speed test query from the CMTS via the second downstream service flow, and transmit a speed test response to the CMTS via the second upstream service flow.

As will be discussed in more detail below, in some embodiments, service program 528 includes instructions that may be used by controller 516 to cause cable modem 412 to receive the first downstream service flow; transmit the first upstream data to the CMTS via the first upstream service flow to be provided to the external network; receive the second downstream service flow from the CMTS; transmit a speed test query to the modem test server via the CMTS and via the second upstream service flow; and receive a speed test response from the modem test server via the CMTS and via the second downstream service flow.

In some of these embodiments, as will be discussed in more detail below, service program 528 includes instructions that may be used by controller 516 to additionally cause cable modem 412 to transmit a speed test response to the CMTS via the second upstream service flow.

As will be described in greater detail below, in some embodiments, service program 528 includes instructions that may be used by controller 516 to additionally cause cable modem 412 to transmit a speed test initiation request to the CMTS via the first upstream service flow.

As will be described in greater detail below, in some embodiments, service program 528 includes instructions that may be used by controller 516 to additionally cause cable modem 412 to transmit a speed test query and receive a speed test response.

As will be described in greater detail below, in some embodiments, service program 528 includes instructions that may be used by controller 516 to additionally cause cable modem 412 to enable modem test server 404 to determine a speed test result based on a speed test query and a speed test response.

As will be described in greater detail below, in some embodiments, service program 528 includes instructions that may be used by controller 516 to additionally cause cable modem 412 to enable modem test server 404 to transmit a speed test result to CMTS 402.

Each of radios 518, 520 and 522 may include one or more antennas to communicate wirelessly via one or more of the 2.4 GHz band, the 5 GHz band, the 6 GHz band, and the 60 GHz band, or at the appropriate band and bandwidth to implement any IEEE 802.11 Wi-Fi protocols, such as the Wi-Fi 4, 5, 6, or 6E protocols. Cable modem 412 can also be equipped with a radio transceiver/wireless communication circuit to implement a wireless connection in accordance with any Bluetooth protocols, Bluetooth Low Energy (BLE), or other short range protocols that operate in accordance with a wireless technology standard for exchanging data over short distances using any licensed or unlicensed band such as the CBRS band, 2.4 GHz bands, 5 GHz bands, 6 GHz bands, or 60 GHz bands, RF4CE protocol, ZigBee protocol, Z-Wave protocol, or IEEE 802.15.4 protocol.

As shown in FIG. 5, CMTS 402 connects with cable modem 412 by way of network node 110. Network node 110 provides connections to multiple cable modems in the network; a non-limiting example of which is cable modem 412.

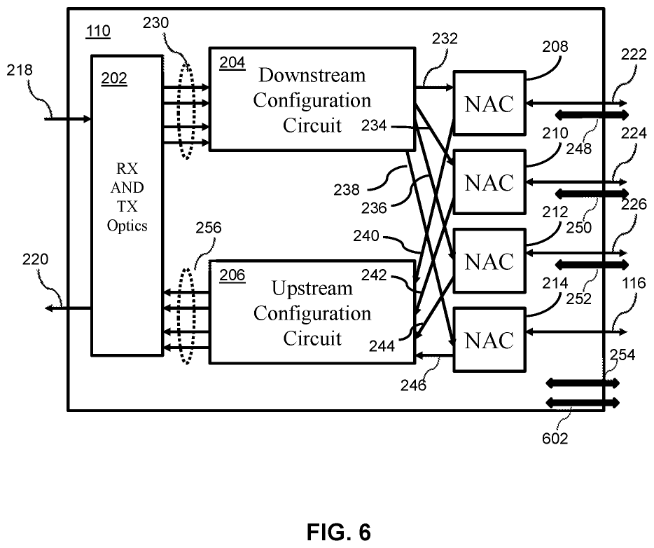

FIG. 6 illustrates an exploded view of network node 110 at time t3.

As shown in FIG. 6, network node 110 includes: an optical communication component 202, a downstream configuration circuit 204, an upstream configuration circuit206, and a network access component (NAC) 208, a NAC 210, a NAC 212, and a NAC 214. Network node 110 communicates with CMTS 402 via downstream service flow 218 and upstream service flow 220. For simplicity, downstream service flow 218 and upstream service flow 220 are shown as a communication line 118 in FIG. 4A.

In operation, at time t3, optical communication component 202 receives the optical service group provider downstream data signals transmitted by CMTS 402 via downstream service flow 218. Once received, optical communication component 202 de-multiplexes the signal from the single optical signal into separate optical signals that are then converted into RF signals. In this example embodiment, the optical signal received contains a wavelength. At this time, optical communication component 202 will transmit the wavelength as an RF signal via downstream service flow communication channel 230 to downstream configuration circuit 204.

In conventional hybrid fiber coax (HFC) network nodes, the configuration between service flows and home networks is hardwired, and as such, downstream configuration circuit 204 must transmit the RF signals to each of NAC 208, NAC 210, NAC 212 and NAC 214 based on the current configuration of network node 110.

Each of NACs 208, 210, 212 and 214 provide data over RF signals in the downstream direction to respective home networks at the correct power level. Similarly, each of NACs 208, 210, 212 and 214 provide data over RF signals in the upstream direction from the respective home networks at the correct power level.

The network node 110 is in a 1×1 configuration since there is just one downstream service flow and one upstream service flow, and data from one service flow is transmitted to network node 110. In this example embodiment, the downstream service flow is transmitted as RF signal 232 to NAC 208, as RF signal 234 to NAC 210, as RF signal 236 to NAC 212, and as RF signal 238 to NAC 214.

After being received, NAC 208 will transmit RF signal 232 to subscriber 222, NAC 210 will transmit RF signal 234 to subscriber 224, NAC 212 will transmit RF signal 236 to subscriber 226, and NAC 214 will transmit RF signal 238 to communication line 116 (to residence 406 in FIG. 4B).

Simultaneously, data being transmitted by each home network will be received by the home networks' corresponding NAC and then transmit as an RF signal to upstream configuration circuit 206. Upstream configuration circuit 206 will then combine the data as configured and transmit the received data as RF signals to optical communication component 202 based on its current configuration. Continuing the above example, since network node 110 is in a 1×1 configuration, upstream configuration circuit 206 will combine the four RF signals from service flow 248, 250, 252, and 254 as RF signals 240, 242, 244 and 246 into a single RF signal and transmit the resulting RF signal data as a single upstream service flow data on an upstream service flow communication channel 256 to optical communication component 202. Once received, optical communication component 202 will convert and transmit the data to CMTS 402 as a single optical signal via upstream service flow 220.

At the subscriber connection level, each NAC provides a separate service flow for each subscriber. For instance, NAC 208 provides service flow 248 to subscriber 222, NAC 210 provides service flow 250 to subscriber 224, and NAC 212 provides service flow 252 to subscriber 226.

As shown in FIG. 6, NAC 214 provides service flow 254 and 602. At this point, there are two service flows: service flow 254, which represents downstream service flow 122 and upstream service flow 124 (as shown in FIGS. 4A and 4B), and service flow 602, which represents downstream service flow 401 and an upstream service flow 403, which will be described in greater detail with reference to FIG. 4B-C.

Returning to the example above, rather than increasing the bandwidth of service flow 254 for the internet speed test, a separate service flow, service flow 602, is created. This is beneficial for both the home network internet, as well as the speed test. Home network traffic no longer has to compete with the speed test, which interrupts the service of all devices in the home, in this case being client device 414. Further, an accurate representation of the speed of the internet can be found, as the speed test is conducted through service flow 602, rather than going through communication channels 120, 118, and 116 as the home network traffic does.

Returning to FIG. 3, after a second service flow is initiated (S306), the speed test is initiated (S308). For example, returning to FIG. 4B, after service flow 602 is initiated, the speed test for residence 406 is initiated. The speed test can now accurately determine the speed of the home network of residence 406 without interrupting the connection of client device 414. Modem test server 404 will send a speed test query down through downstream service flow 401.

Returning to FIG. 3, after the speed test is initiated (S308), the speed test is completed (S310). This will be described in greater detail with reference to FIG. 4C.

FIG. 4C illustrates structural components implementing a communication infrastructure 400 at a time t₂ .

In addition to the components of FIGS. 4A and 4B, FIG. 4C also includes an upstream 403, which is distinct from upstream 124.

Returning to FIG. 3, after the speed test is completed (S310), the second service flow is terminated (S312). For example, with reference to FIG. 4C, after modem test server 404 sends a speed test query down through downstream service flow 401, the speed test response is sent up through upstream service flow 403. This concludes the speed test for residence 406. Once the speed test is complete, service flow 602 will be terminated.

After the second service flow is terminated (S312), method 300 stops (S314).

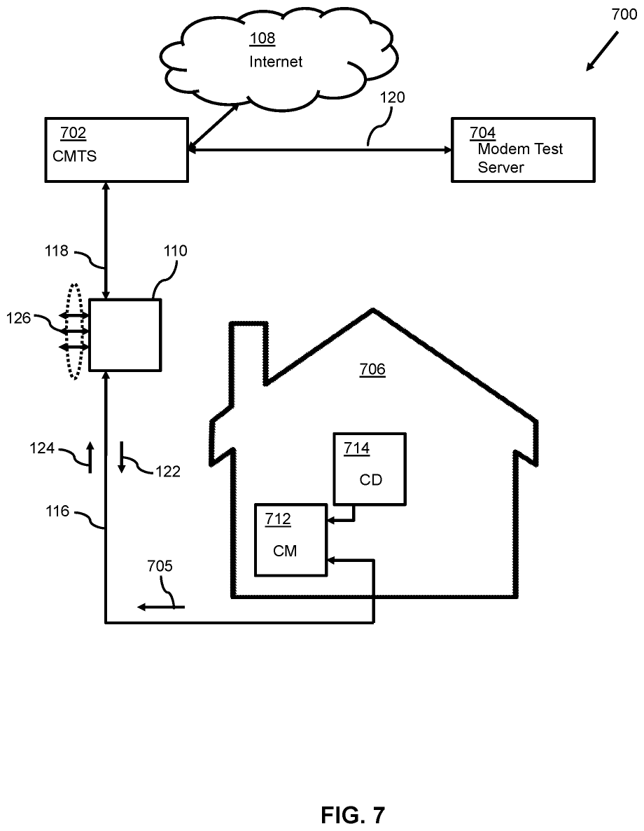

In some embodiments, the speed test may be requested by a user through a cable modem, rather than having the speed test initiated by the modem test server. This will be described with greater detail in reference to FIG. 7.

FIG. 7 illustrates structural components implementing a communication infrastructure 700 at a time t₅.

As shown in the figure, communication infrastructure 700 includes a CMTS 702, a modem test server 704, a speed test request 705, a residence 706 which includes one cable modem 712 and a client device 714, internet 108, a network node 110, communication channels 116, 118, and 120, downstream service flow 122, upstream service flow 124, and a plurality of service flows 126. Cable modem 712 can communicate with modem test server 704 by way of network node 110 and CMTS 702. Network node 110 provides a plurality of service flows 126 to multiple home networks.

For example, assume that a resident (not shown) in residence 706 uses cable modem 712 to request that a speed test be performed. Rather than modem test server 704 initiating the speed test, cable modem 712 sends speed test request 705 to CMTS 702. Then, the speed test process would be run in a similar manner to the example embodiment discussed above with reference to FIGS. 4A-6.

In the non-limiting example embodiments discussed above, modem test server 404 transmits the speed test query, receives the speed test response and determines the speed test result based on the speed test query and the speed test response. However, in some embodiments, the cable modem may transmit the speed test query, receive the speed test response and determine the speed test result based on the speed test query and the speed test response.

For example, in a modification to the non-limiting example embodiment discussed above with respect to FIG. 7, in some embodiments, a resident (not shown) in residence 706 uses cable modem 712 to request that a speed test be performed. Rather than modem test server 704 initiating the speed test, cable modem 712 sends speed test request 705 to CMTS 702. Then, as shown in FIG. 7, CMTS 702 would provide a second upstream service flow and a second downstream service flow to cable modem 712.

However, in this embodiment, the controller (not shown) in cable modem 712 would execute instructions to cause cable modem 712 to transmit a speed test query to modem test server 704 via the newly provided second upstream service flow. Modem test server 704 would then provide a speed test response to cable modem 712 via the newly provided second downstream service flow.

The controller (not shown) in cable modem 712 would execute additional instructions to cause cable modem 712 to determine a speed test result based on the speed test query that cable modem 712 transmitted and the speed test response provided by modem test server 704. Therefore, in this embodiment, cable modem 712 is controlling the speed test and determining the speed test result.

A CMTS will typically run automated speed tests to measure the downlink and uplink speeds to each client. The “speed” is a measure of how long it takes to download or upload a certain number of bytes in a specific time frame. In a conventional speed test, the CMTS run the tests on the customer data flows impacting the customer services partially and sometimes fully.

In the non-limiting example embodiments discussed above, a second upstream service flow and a second downstream service flow are provided to the cable modem for purposes of servicing a speed test. However, it should be noted that multiple distinct upstream and downstream service flows may be provided to a cable modem, wherein any one of the multiple upstream and downstream service flows may be used to service a speed test in accordance with aspects of the present disclosure.

In accordance with the present disclosure, a second data flow is created, parallel to the preexisting customer data flow. The first data flow contains the user data and the second data flow is used solely for conducting the speed test during normal operation of the cable modem. The CMTS measures the speed using this additional data flow. This way, the customer data flow is not impacted and the speed can still be measured. A speed test query is sent from the modem test server through the CMTS to the cable modem, and then a speed test response is sent back to the modem test server. When the speed test is concluded, the second data flow will be terminated.

The present disclosure as disclosed will accurately measure the speed of a customer's home network, as the customer data flow will not be slowed down to incorporate the speed test as well. Further, the customer will not notice a drop in internet efficiency when the second data flow is used to conduct the speed test.

The operations disclosed herein may constitute algorithms that can be effected by software, applications (apps, or mobile apps), or computer programs. The software, applications, computer programs can be stored on a non-transitory computer-readable medium for causing a computer, such as the one or more processors, to execute the operations described herein and shown in the drawing figures.

The foregoing description of various preferred embodiments have been presented for purposes of illustration and description. It is not intended to be exhaustive or to limit the present disclosure to the precise forms disclosed, and obviously many modifications and variations are possible in light of the above teaching. The example embodiments, as described above, were chosen and described in order to best explain the principles of the present disclosure and its practical application to thereby enable others skilled in the art to best utilize the present disclosure in various embodiments and with various modifications as are suited to the particular use contemplated. It is intended that the scope of the present disclosure be defined by the claims appended hereto.

What is claimed is:

1. A cable modem termination system (CMTS) for use with a modem test server, an external network, and a cable modem, the modem test server being configured to transmit a speed test initiation signal and a speed test query, the cable modem being configured to transmit a speed test response, said CMTS comprising:
a memory; and
a processor configured to execute instructions stored on said memory to cause said CMTS to:
provide a first downstream service flow to the cable modem, the first downstream service flow including first downstream data from the external network;
provide a first upstream service flow to the cable modem, the first upstream service flow including first upstream data to be provided to the external network;
receive the speed test initiation request from the modem test server;
provide a second downstream service flow to the cable modem, the second downstream service flow being distinct from the first downstream service flow;
provide a second upstream service flow to the cable modem, the second upstream service flow being distinct from the first upstream service flow;
receive the speed test query from the modem test server;
transmit the speed test query to the cable modem via the second downstream service flow;
receive the speed test response from the cable modem via the second upstream service flow; and
transmit the speed test response to the modem test server.

2. The CMTS of claim 1, wherein said processor is configured to execute instructions stored on said memory to additionally cause said CMTS to stop providing the second downstream service flow and the second upstream service flow to the cable modem after the speed test response is transmitted to the modem test server.

3. The CMTS of claim 2, wherein said processor is configured to execute instructions stored on said memory to additionally cause said CMTS to transmit a speed test result to the cable modem via the first downstream service flow.

4. The CMTS of claim 1, wherein said processor is configured to execute instructions stored on said memory to additionally cause said CMTS to transmit a speed test result to the cable modem via the second downstream service flow.

5. A method of using a cable modem termination system (CMTS) with a modem test server, an external network, and a cable modem, the modem test server being configured to transmit a speed test initiation signal and a speed test query, the cable modem being configured to transmit a speed test response, said method comprising:
providing, via a processor configured to execute instructions stored on a memory, a first downstream service flow to the cable modem, the first downstream service flow including first downstream data from the external network;
providing, via the processor, a first upstream service flow to the cable modem, the first upstream service flow including first upstream data to be provided to the external network;
receiving, via the processor, the speed test initiation request from the modem test server;
providing, via the processor, a second downstream service flow to the cable modem, the second downstream service flow being distinct from the first downstream service flow;
providing, via the processor, a second upstream service flow to the cable modem, the second upstream service flow being distinct from the first upstream service flow;
receiving, via the processor, the speed test query from the modem test server;
transmitting, via the processor, the speed test query to the cable modem via the second downstream service flow;
receiving, via the processor, the speed test response from the cable modem via the second upstream service flow; and
transmitting, via the processor, the speed test response to the modem test server.

6. The method of claim 5, further comprising stop providing, via the processor, the second downstream service flow and the second upstream service flow to the cable modem after the speed test response is transmitted to the modem test server.

7. The method of claim 6, further comprising transmitting, via the processor, a speed test result to the cable modem via the first downstream service flow.

8. The method of claim 5, further comprising transmitting, via the processor, a speed test result to the cable modem via the second downstream service flow.

9. A non-transitory, computer-readable media having computer-readable instructions stored thereon, the computer-readable instructions being capable of being read by a cable modem termination system (CMTS) for use with a modem test server, an external network, and a cable modem, the modem test server being configured to transmit a speed test initiation signal and a speed test query, the cable modem being configured to transmit a speed test response, wherein the computer-readable instructions are capable of instructing the CMTS to perform the method comprising:
providing, via a processor configured to execute instructions stored on a memory, a first downstream service flow to the cable modem, the first downstream service flow including first downstream data from the external network;
providing, via the processor, a first upstream service flow to the cable modem, the first upstream service flow including first upstream data to be provided to the external network;
receiving, via the processor, the speed test initiation request from the modem test server;
providing, via the processor, a second downstream service flow to the cable modem, the second downstream service flow being distinct from the first downstream service flow;
providing, via the processor, a second upstream service flow to the cable modem, the second upstream service flow being distinct from the first upstream service flow;
receiving, via the processor, the speed test query from the modem test server;
transmitting, via the processor, the speed test query to the cable modem via the second downstream service flow;
receiving, via the processor, the speed test response from the cable modem via the second upstream service flow; and
transmitting, via the processor, the speed test response to the modem test server.

10. The non-transitory, computer-readable media of claim 9, wherein the computer-readable instructions are capable of instructing the CMTS to perform the method further comprising stop providing, via the processor, the second downstream service flow and the second upstream service flow to the cable modem after the speed test response is transmitted to the modem test server.

11. The non-transitory, computer-readable media of claim 10, wherein the computer-readable instructions are capable of instructing the CMTS to perform the method further comprising transmitting, via the processor, a speed test result to the cable modem via the first downstream service flow.

12. The non-transitory, computer-readable media of claim 9, wherein the computer-readable instructions are capable of instructing the CMTS to perform the method further comprising transmitting, via the processor, a speed test result to the cable modem via the second downstream service flow.

13. A cable modem for use with a cable modem termination system (CMTS), a modem test server, and an external network, the modem test server being configured to transmit a speed test query, the CMTS being configured to provide a first downstream service flow including first downstream data from the external network, to provide a second downstream service flow including the speed test query, to provide a first upstream service flow including first upstream data to be provided to the external network, and to provide a second upstream service flow, said cable modem comprising:
a memory; and
a processor configured to execute instructions stored on said memory to cause said cable modem to:
receive the first downstream service flow;
transmit the first upstream data to the CMTS via the first upstream service flow to be provided to the external network;
transmit a speed test initiation request to the CMTS via the first upstream service flow;
receive the second downstream service flow from the CMTS;
receive the speed test query from the CMTS via the second downstream service flow; and
transmit a speed test response to the CMTS via the second upstream service flow.

14. A method of using a cable modem with a cable modem termination system (CMTS), a modem test server, and an external network, the modem test server being configured to transmit a speed test query, the CMTS being configured to provide a first downstream service flow including first downstream data from the external network, to provide a second downstream service flow including the speed test query, to provide a first upstream service flow including first upstream data to be provided to the external network, and to provide a second upstream service flow, said method comprising:
receiving, via a processor configured to execute instructions stored on a memory, the first downstream service flow;
transmitting, via the processor, the first upstream data to the CMTS via the first upstream service flow to be provided to the external network;
transmitting, via the processor, a speed test initiation request to the CMTS via the first upstream service flow;
receiving, via the processor, the second downstream service flow from the CMTS;
receiving, via the processor, the speed test query from the CMTS via the second downstream service flow; and
transmitting, via the processor a speed test response to the CMTS via the second upstream service flow.

15. A non-transitory, computer-readable media having computer-readable instructions stored thereon, the computer-readable instructions being capable of being read by a cable modem for use with a cable modem termination system (CMTS), a modem test server, and an external network, the modem test server being configured to transmit a speed test query, the CMTS being configured to provide a first downstream service flow including first downstream data from the external network, to provide a second downstream service flow including the speed test query, to provide a first upstream service flow including first upstream data to be provided to the external network, and to provide a second upstream service flow, wherein the computer-readable instructions are capable of instructing the cable modem to perform the method comprising:
receiving, via a processor configured to execute instructions stored on a memory, the first downstream service flow;
transmitting, via the processor, the first upstream data to the CMTS via the first upstream service flow to be provided to the external network;
transmitting, via the processor, a speed test initiation request to the CMTS via the first upstream service flow;
receiving, via the processor, the second downstream service flow from the CMTS;
receiving, via the processor, the speed test query from the CMTS via the second downstream service flow; and
transmitting, via the processor a speed test response to the CMTS via the second upstream service flow.

16. A cable modem for use with a cable modem termination system (CMTS), a modem test server, and an external network, the CMTS being configured to provide a first downstream service flow including first downstream data from the external network, to provide a second downstream service flow, to provide a first upstream service flow including first upstream data to be provided to the external network, and to provide a second upstream service flow, said cable modem comprising:
a memory; and
a processor configured to execute instructions stored on said memory to cause said cable modem to:
receive the first downstream service flow;
transmit the first upstream data to the CMTS via the first upstream service flow to be provided to the external network;
receive the second downstream service flow from the CMTS;
transmit a speed test query to the modem test server via the CMTS and via the second upstream service flow; and
receive a speed test response from the modem test server via the CMTS and via the second downstream service flow.

17. The cable modem of claim 16, wherein said processor is configured to execute instructions stored on said memory to additionally cause said cable modem to transmit a speed test response to the CMTS via the second upstream service flow.

18. The cable modem of claim 16, wherein said processor is configured to execute instructions stored on said memory to additionally cause said cable modem to transmit a speed test initiation request to the CMTS via the first upstream service flow.

19. A method of using a cable modem with a cable modem termination system (CMTS), a modem test server, and an external network, the CMTS being configured to provide a first downstream service flow including first downstream data from the external network, to provide a second downstream service flow, to provide a first upstream service flow including first upstream data to be provided to the external network, and to provide a second upstream service flow, said method comprising:
receiving, via a processor configured to execute instructions stored on a memory, the first downstream service flow;
transmitting, via the processor, the first upstream data to the CMTS via the first upstream service flow to be provided to the external network;
receiving, via the processor, the second downstream service flow from the CMTS;
transmitting, via the processor a speed test query to the modem test server via the CMTS and via the second upstream service flow; and
receiving, via the processor, a speed test response from the modem test server via the CMTS and via the second downstream service flow.

20. The method of claim 19, further comprising transmitting, via the processor, a speed test response to the CMTS via the second upstream service flow.

21. The method of claim 19, further comprising transmitting, via the processor, a speed test initiation request to the CMTS via the first upstream service flow.

22. A non-transitory, computer-readable media having computer-readable instructions stored thereon, the computer-readable instructions being capable of being read by a cable modem for use with a cable modem termination system (CMTS), a modem test server, and an external network, the CMTS being configured to provide a first downstream service flow including first downstream data from the external network, to provide a second downstream service flow, to provide a first upstream service flow including first upstream data to be provided to the external network, and to provide a second upstream service flow, wherein the computer-readable instructions are capable of instructing the cable modem to perform the method comprising:
receiving, via a processor configured to execute instructions stored on a memory, the first downstream service flow;
transmitting, via the processor, the first upstream data to the CMTS via the first upstream service flow to be provided to the external network;
receiving, via the processor, the second downstream service flow from the CMTS;
transmitting, via the processor a speed test query to the modem test server via the CMTS and via the second upstream service flow; and
receiving, via the processor, a speed test response from the modem test server via the CMTS and via the second downstream service flow.

23. The non-transitory, computer-readable media of claim 22, wherein the computer-readable instructions are capable of instructing the cable modem to perform the method further comprising transmitting, via the processor, a speed test response to the CMTS via the second upstream service flow.

24. The non-transitory, computer-readable media of claim 22, wherein the computer-readable instructions are capable of instructing the cable modem to perform the method further comprising transmitting, via the processor, a speed test initiation request to the CMTS via the first upstream service flow.
